(19) United States
(12) Patent Application Publication
(10) Pub. No.: US 2015/0112451 A1
(43) Pub. Date: Apr. 23, 2015

(54) ULTRASOUND SYSTEM FOR REAL-TIME TRACKING OF MULTIPLE, IN-VIVO STRUCTURES

(71) Applicants: Nikolai Dechev, Victoria (CA); Kelly Stegman, Victoria (CA)

(72) Inventors: Nikolai Dechev, Victoria (CA); Kelly Stegman, Victoria (CA)

(21) Appl. No.: 14/058,022

(22) Filed: Oct. 18, 2013

(51) Int. Cl. A61F 2/54; A61B 8/00

(52) U.S. Cl. CPC A61F 2/54; A61B 8/42; A61F 2002/543
USPC 623/63; 600/447

(57) ABSTRACT

An ultrasound tracking system for tracking shallow structures by acquiring and processing a sequence of images is provided. The system comprises a transducer, a beamformer, and computational processing hardware, wherein the transducer has a plurality of sub-arrays with a gap between adjacent sub-arrays, the sub-arrays in generally parallel relation to one another, the sub-arrays comprising at least 12 elements, the beamformer in electronic communication with the sub-arrays, and the computational processing hardware comprising instructions for transforming signals from the sub-arrays into a plurality of data sets. An active hand prosthesis and an active hand exoskeleton is also provided.

### FIELD

The present technology relates to a system and method for tracking movement of internal structures. More specifically, the technology relates to an ultrasound tracking system for locating in the vicinity of a user's wrist or lower arm that can track movement of the tendons of the hand.

### BACKGROUND

Various methods of tracking movement of body parts have been developed. A number of these are based on measuring forces exerted on the body parts. For example, Elisa Morgantia et al (International Symposium on Robotics and Intelligent Sensors 2012 (IRIS 2012) used piezoresistive-based sensors to detect the force exerted by the tendons in different configurations of the hand and of the wrist.

Ren et al (Appl Opt. 2007 Oct. 1;46 (28):6867-71) implanted Fiber Bragg grating displacement sensors for movement measurement of tendons and ligaments. The sensors measure strain in the tendons and ligaments, and this information is then transformed into a measure of movement.

Ultrasound has been used extensively to image internal body parts. The transducer is often designed for a specific application, and therefore there are many transducer designs. In general, the transducer head has an array of elements. The arrays may be arranged in sparse arrays or subarrays. For example, US Publication No. 20130102902 relates to an ultrasound imaging system in which the scan head either includes a beamformer circuit that performs far field subarray beamforming or includes a sparse array selecting circuit that actuates selected elements. When used with second stage beamforming system, three dimensional ultrasound images can be generated.

Subarrays have been used to generate three-dimensional images. For example, subarrays may be used in US Publication No. 20050288588, to obtain real-time 3D ultrasonic images. The method and apparatus for electronic volume data acquisition using ultrasound generates image data in a scanning and imaging process is known as coherent aperture combining beamforming (CAC-BF). The CAC-BF technique can be applied in an azimuth dimension and/or an elevation dimension, to form an ultrasound image line, image plane, or image data cube. Several innovations relating to the design and ordering of shots and efficient weighting algorithms that address various performance issues associated with B-mode and other modes such as Doppler, and harmonic imaging are disclosed. The invention has significant advantages over other synthetic aperture imaging techniques and conventional imaging techniques by supporting higher resolution, larger volumes and/or shorter acquisition times than comparative techniques, using similar system hardware complexity.

Subarrays also enable the use of steered and focused beams and the estimation of different aberration values for different regions of tissue, as well as improved signal-to-noise. In US Publication No. 20050148874, this is done by steering a receive subarray to an image region for which aberration data is to be obtained. Transmission is now done over a range of transmit steering angles to fully cover the region within the receive beam profile.

Grönlund et al (Ultrasound Med Biol. 2013 Feb.;39 (2):360-9.) used two dimensional ultrasound to study mechanical waves of skeletal muscle contraction. B mode image acquisition during multiple consecutive electrostimulations, speckle tracking and a time-stamp sorting protocol were used to obtain 2D tissue velocity imaging of biceps brachii muscle contraction. They were able to demonstrate that 2D mechanical wave imaging provides simultaneous assessment of active and passive muscle tissue properties. However, 2D ultrasound tracking has inherent shortcomings: it requires that the speckle patterns remain in the 2D imaging plane; and the lack of 3D information makes tracking impossible in the off-plane direction and thus limits both the robustness and accuracy of the tracking algorithm.

If speckle tracking is used, a specific location on the moving tissue itself is tracked. This means the ROI changes position (follows the tissue) across the screen, during the B-Scan image sequence. As well, only the original Template from frame t is used for comparison to all subsequent image frames. Therefore, tracking can be easily lost if the matching TempBox was actually incorrect, and then used as the next Template.

US Publication No. 20110237949 is a system and method for using dynamic ultrasonic imaging to analyze a subject's carpal tunnel and generate risk factors indicative of the health of the subject's subsynovial connective tissue and the subject's risk of developing carpal tunnel syndrome. The system and method uses speckle imaging techniques to track dynamic structures within the carpal tunnel and statistical analysis techniques to compare the properties of these dynamic structures of the subject to those of normal subjects and subjects having carpal tunnel syndrome.

US Publication No. 20090221916 is directed to methods for obtaining information about the mechanical behaviour of structures associated with mammalian joints and tendons. Embodiments of such methods include creating deformation in a joint structure (such as ligaments and articular cartilage) or tendon of interest, using an ultrasound scanner and a single element or array of elements to acquire sequences of ultrasound data of the joint structure or tendon, estimating one, two or three components of the resulting displacement and strain between a reference frame of ultrasound data and successive frames of ultrasound data, and using a cross-correlation algorithm to estimate the displacement and strain components. This information may be used to inform the design of tissue grafts. Tissue grafts produced using this information are also provided. The same method can be used in situ together with non-invasive or invasive procedures. The method is suited to measuring displacement and strain of a single tendon, when put under mechanical load.

Tendon motion, in terms of displacement and velocity, has a direct kinematic correlation to the biomechanical motion of the hand digits. In order to exploit this relationship, a system is needed that can accurately track movement of tendons relative to the surrounding tissue, by acquiring internal tissue images, and transforming the images into useable tendon displacement data. Ultrasound systems provide such potential; however, the present systems are unsuitable for continuous monitoring and real-time tracking of tendon displacement for portable/wearable applications. In particular, current ultrasound systems are too expensive, and cumbersome in size for such applications. Additionally, current ultrasound systems have general-purpose array transducers that cannot track multiple tendons simultaneously, and are too slow to acquire the image-frame frequency needed for good tendon tracking. In addition, current ultrasound systems lack the software needed for real-time tracking of tendon displacement.

The ultrasound tracking system would preferably include: a portable-sized hardware that is wearable by a person, where that hardware includes the transducer, the beamformer, the computational processing hardware, and a power source. Additionally, it would include software capable of data transformation with low computational intensity that can be handled by the computational hardware to provide real-time tracking. The ultrasound transducer should be small and easy to use.

Prosthetic systems for the hand are wearable devices worn on the distal end of the amputated limb. They serve to replace the function lost due to a hand deficiency. Exoskeleton systems for the hand are wearable gauntlet-like devices that supplement the function of a healthy hand to improve strength. There are many prosthetic hand designs, and many exoskeleton designs. Historically, artificial prosthetic limbs were used as a supplement for balance and cosmetic purposes. Today's prosthetics are advanced mechanical systems, allowing for functional articulations and improved strength. Hand prosthesis for example, are usually comprised of individual articulating finger joints, an opposable thumb and a rotational wrist, allowing for a multi-degree-of-freedom device. Some examples of available prosthetic devices include those from Touch Bionics® and OttoBock®. These have motor driven digits. US Publication No. 20120146352 to OttoBock is a gripping device, comprising a proximal member, a medial member, and a distal member (phalanges), which are each pivotably supported on each other, and comprising an actuator, which is a motor that is coupled to a slidably supported coupling element, wherein the coupling element is arranged between the proximal member and the distal member and is connected in a force-transmitting manner both to the proximal member and to the distal member. According to the invention, at least one lever is arranged on the coupling element, and the lever is connected both to the proximal member and to the distal member and kinematically couples the proximal member with the distal member.

U.S. Pat. No. 5,888,246 is to a motor drive system and linkage for a hand prosthesis. The hand prosthesis of U.S. Pat. No. 5,888,246 has at least one motor driven digit with the digit moving around an axis to thereby achieve flexion and extension.

Some devices have thumb rotation. US Publication No. 20130046395 is to a hand prosthesis including a hand chassis, a thumb member mounted on the hand chassis for rotation of the thumb member in relation to the hand chassis about an axis extending generally along the length of the thumb member, a motor disposed on one of the hand chassis and the thumb member, the motor being operable to drive a worm and a worm gear wheel disposed on the other of the hand chassis and the thumb member, the worm being in engagement with the worm gear wheel such that, upon operation of the motor, the thumb member rotates in relation to the hand chassis. US Publication No. 20070213842 similarly has thumb rotation.

Even though there exist several anatomically correct and sophisticated multi-degree-of-freedom prosthetic hands, there are few or no current ways of detecting “user intention” to fully control may or all degrees-of-freedom on these prostheses. User-intention refers to the ability for computational hardware and software to detect the user's intent for motion. Such prostheses are typically controlled by surface electromyography (EMG) signals detect the user's intent for motion, but problems due to sensor crosstalk and spatial resolution limit surface EMG sensory systems to measure one or two independent signals. This provides limited functionality, in comparison to less complex non-electric prosthesis.

A prosthetic hand that can be controlled by tracking tendon movement with ultrasound is needed. That tendon movement information is preferably relayed to microprocessors, which in turn actuate motors to effect movement in four degrees of freedom—the fingers in flexion and extension, the thumb in flexion and extension and the thumb in adduction and abduction.

### SUMMARY

An ultrasound tracking system is provided that comprises a transducer, a beamformer, computational processing hardware, and software. The ultrasound tracking system can accurately track movement of one or more tendons relative to stationary tissue and transform the images into useable data. In certain configurations, the system has redundancy in tracking for higher accuracy. High quality, focused imaging of structures in the vicinity of the surface is possible. The transducer is small and easy to use. The computational processing hardware is portable, and contains software capable of data transformation with low computational intensity, that can be handled by the computational processing hardware to provide real-time tracking.

In one embodiment an ultrasound tracking system for tracking internal structures by acquiring and processing two-dimensional images is provided. The system comprises a transducer, a beamformer, and computational processing hardware, wherein the transducer has a plurality of sub-arrays with a gap between adjacent sub-arrays, the sub-arrays in generally parallel relation to one another, the sub-arrays comprising at least 12 elements, the beamformer in electronic communication with the sub-arrays, and the computational processing hardware comprising instructions for transforming signals from the sub-arrays into a plurality of data sets.

In the ultrasound tracking system, there may be at least three sub-arrays, each comprising at least about 16 elements.

In the ultrasound tracking system, the elements may have a pitch of no more than about 300 microns.

In the ultrasound tracking system, the gap between adjacent sub-arrays may be less than about 3 millimeters.

In the ultrasound tracking system, there may be four sub-arrays, each comprising 32 elements.

In the ultrasound tracking system, the transducer may further comprise at least two circuit boards, the circuit boards being offset to provide a compact transducer.

The ultrasound tracking system may further comprise a cable for communication between the transducer and the computational processing hardware, the cable extending normal to a proximal end of the transducer.

In the ultrasound tracking system, the beamformer and computational processing hardware may further comprise instructions for sequential firing of the elements.

In the ultrasound tracking system, the beamformer and computational processing hardware may further comprise instructions for measuring a stationary region of interest.

The ultrasound tracking system may further comprise a display.

In another embodiment, a transducer is provided for use with an ultrasound system, the transducer comprising: at least two sub-arrays in parallel relation to define an at least one gap between the sub-arrays, the sub-arrays comprising an at least 12 elements, the elements having a pitch of at most about 300 microns; an at least two circuit boards; a housing for housing the sub-arrays and the circuit boards; and a connector for connecting a cable, the connector located on a proximal side of the housing and extending normal to the proximal side.

The transducer may comprise four sub-arrays in generally parallel relation to define three gaps of at most about 3 mm and four circuit boards, the circuit boards being offset.

In the transducer, the sub-arrays may comprise 32 elements.

In another embodiment a method of shallow imaging an at least one internal, moving structure is provided, the method comprising: placing a transducer onto a surface adjacent a region of interest, wherein the transducer has a plurality of sub-arrays with a gap between adjacent sub-arrays, the sub-arrays in generally parallel relation to one another, the sub-arrays comprising an at least 12 elements; driving the transducer with a beamformer and computational processing hardware; the transducer scanning and sending signals from each sub-array; and the computational processing hardware collecting signals from each sub-array and transforming the signals into a plurality of data sets.

In the method, scanning may be at a rate of at least 30 frames per second.

In the method, scanning may comprise firing a burst of elements in a variable aperture.

The method may further comprise redundant imaging to provide redundant image data the computational processing hardware identifying and processing the redundant image data.

In the method, the region of interest may be a stationary region of interest.

In the method, the at least one internal, moving structure may be an at least one tendon.

In the method, the at least one internal, moving structure may be three tendons in a carpel tunnel.

A method of tracking movement of an internal structure is also provided, the method comprising: collecting a first ultrasound image of the structure and determining a region of interest on the structure; collecting at least a second ultrasound image of the region of interest; and calculating a displacement of the region of interest, thereby tracking movement of the shallow structure.

The method may comprise collecting a series of ultrasound images of the region of interest and calculating total displacement of the region of interest.

In the method, a transducer may be utilized for the collecting, the transducer comprising a plurality of sub-arrays with a gap between adjacent sub-arrays, the sub-arrays in generally parallel relation to one another, the sub-arrays comprising an at least 12 elements.

The method for tracking movement of an at least two internal structures may further comprise aligning the sub-arrays over the at least two shallow structures before collecting the ultrasound images.

In the method, the internal structures may be tendons.

In another embodiment, an active hand prosthesis to provide four degrees of freedom is provided. The active hand prosthesis comprises a hand prosthesis and a controller, the hand prosthesis comprising: at least an index finger, a middle finger, and a ring finger, each finger comprising: a proximal phalanx, an intermediate phalanx and a distal phalanx, each hinged at an interphalangeal joint; and linkages to an actuator; a thumb comprising: a metacarpal; a proximal phalanx; and a distal phalanx; the proximal phalanx and the distal phalanx hinged at an interphalangeal joint, the metacarpal pivotally attached to a palm plate by a pivot assembly, the pivot assembly in communication with a pivot assembly actuator, to provide adduction and abduction; and linkages to the index finger actuator; a controller, the controller comprising: a microcontroller, motor amplifiers, the actuators, and a power source; and a tendon tracking system in communication with the microcontroller.

The active hand prosthesis may comprise four actuators, each comprising an electric direct current (DC) motor, an encoder and a gearbox, wherein three actuators: are configured to effect flexion and extension of the index finger, the middle finger, the ring finger, a pinky finger and a thumb; and are each connected to a lead-screw, each lead-screw connected to a slider, and a fourth actuator is connected to the thumb with a cogged belt and pulley system.

In the active hand prosthesis the index finger may be connected to a first common slider that is common with the thumb by a cable to actuate the index finger and thumb together, and the ring finger is connected to a second common slider that is common with the pinky finger, to actuate the ring finger and pinky finger together.

The active hand prosthesis may further comprise pressure sensors and a rotational angle sensor.

In the active hand prosthesis, the tracking system may be an ultrasound tracking system.

In the active hand prosthesis, the ultrasound tracking system may be a sparse array ultrasound tracking system.

In the active hand prosthesis, the sparse array ultrasound tracking system may comprise a transducer, the transducer comprising a plurality of sub-arrays with a gap between adjacent sub-arrays, the sub-arrays in generally parallel relation to one another, the sub-arrays comprising an at least 12 elements.

In the active hand prosthesis, there may be at least three sub-arrays, each comprising at least about 12 elements.

In the active hand prosthesis, the elements may have a pitch of no more than about 300 micron.

In the active hand prosthesis, the gap between adjacent sub-arrays may be less than about 3 millimeters.

In the active hand prosthesis, there may be four sub-arrays, each comprising 32 elements.

In the active hand prosthesis, the transducer may further comprise four circuit boards, the circuit boards being offset to provide a compact transducer.

In the active hand prosthesis, may further comprise a beamformer.

In the active hand prosthesis, the beamformer and the microcontroller may further comprise instructions for sequential firing of the elements.

In the active hand prosthesis, the beamformer and microcontroller may further comprise instructions for measuring a stationary region of interest.

In another embodiment, an active hand prosthesis is provided, the active hand prosthesis comprising a hand prosthesis, a controller and a tracking system, the hand prosthesis comprising a series of digits configured for flexion and extension, a thumb configured for flexion, extension, adduction and abduction, a series of linkages for each digit and the thumb, and actuators to drive the linkages, the controller comprising a microcontroller, a power source and motor amplifiers, the tracking system comprising an ultrasound tracking system comprising a transducer, and a beamformer, wherein the transducer has a plurality of sub-arrays with a gap between adjacent sub-arrays, the sub-arrays in generally parallel relation to one another, the sub-arrays comprising at least 12 elements, and the beamformer in electronic communication with the sub-arrays.

In the active hand prosthesis, the hand prosthesis may comprise: at least an index finger, a middle finger and a ring finger, each finger comprising: a proximal phalanx, an intermediate phalanx and a distal phalanx, each hinged at an interphalangeal joint; and linkages to the actuator; a thumb comprising: a metacarpal; a proximal phalanx; and a distal phalanx; the proximal phalanx and the distal phalanx hinged at an interphalangeal joint, the metacarpal pivotally attached to a palm plate by a pivot assembly, the pivot assembly in communication with a pivot assembly actuator, to provide adduction and abduction; and linkages to the index finger actuator.

The active hand prosthesis may further comprise pressure sensors and a rotational angle sensor.

The active hand prosthesis may comprise four actuators, each comprising an electric direct current (DC) motor, an encoder and a gearbox, wherein three actuators: are configured to effect flexion and extension of four fingers, the index finger, the middle finger, the ring finger, a pinky finger and the thumb; and are each connected to a lead-screw, each lead-screw connected to a slider, and a fourth actuator is connected to the thumb with a cogged belt and pulley system.

In the active hand prosthesis, the index finger may be connected to a first common slider that is common with the thumb by a cable to actuate the index finger and thumb together, and the ring finger is connected to a second common slider that is common with the pinky finger, to actuate the ring finger and pinky finger together.

An active hand exoskeleton is also provided, the active hand exoskeleton comprising a hand exoskeleton, a controller and a tracking system, the hand exoskeleton comprising a series of cuffs, linkages configured for flexion and extension, and control lines, the controller comprising a microcontroller, a power source and motor amplifiers, the tracking system comprising an ultrasound tracking system comprising a transducer, and a beamformer, wherein the transducer has a plurality of sub-arrays with a gap between adjacent sub-arrays, the sub-arrays in generally parallel relation to one another, the sub-arrays comprising at least 12 elements, and the beamformer in electronic communication with the sub-arrays.

The active hand exoskeleton may further comprise pressure sensors.

### FIGURES

FIG. 1 is a schematic of the ultrasound system of the present technology.

FIG. 2 is a view of the transducer of FIG. 1.

FIG. 3 is median cross sectional view of the transducer of FIG. 1.

FIG. 4 shows the sparse array processing steps to collecting a sequence of images of moving tendons, and then tracking the tendons' motion in accordance with the present technology.

FIG. 5 is a prosthetic hand of the present technology.

FIG. 6 is a diagram of the components of the digits of the present technology.

FIG. 7 is an exoskeleton of the present technology.

FIG. 8 is a flow chart of the components of the controller of the present technology.

FIG. 9 is a diagram of the drive components of the prosthetic hand of FIG. 5.

### DESCRIPTION

Except as otherwise expressly provided, the following rules of interpretation apply to this specification (written description, claims and drawings): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, The terms “comprising,” “having,” “including,” and “containing” are to be construed as open-ended terms (i.e., meaning “including, but not limited to,”) unless otherwise noted.

To the extent necessary to provide descriptive support, the subject matter and/or text of the appended claims is incorporated herein by reference in their entirety.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

All methods described herein can be performed in any suitable order unless otherwise indicated herein or otherwise clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

In the context of the present technology, body refers to any part of a mammal and user similarly refers to a mammal. Tendon may be the tendon of any mammal.

In the context of the present technology, plurality refers to two or more.

In the context of the present technology, shallow imaging refers to imaging within the focal zone, at a depth of about 2 mm to about 25 mm and all ranges there between.

In the context of the present technology, scanning refers to acquiring tissue image frames from all sub-arrays.

In the context of the present technology, internal structures are tendons, muscles, artificial tendons, implanted objects within tendons or muscles, or tendon with muscle (i.e. including the junction).

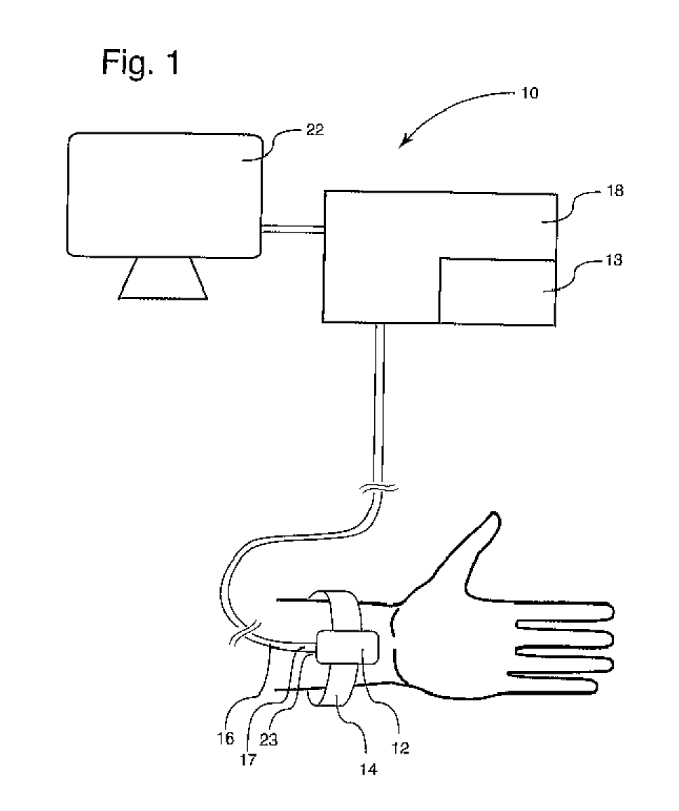

An ultrasound system, generally referred to as 10 is shown in FIG. 1. The system 10 has a transducer 12, that sends and receives the sound waves, a wrist band 14, a coaxial cable 16 for connection with a beamformer 13 and computational hardware 18 that performs calculations, stores data in its memory and contains the electrical power supplies for itself and the transducer 12, transducer pulse controls within the beamformer 13 that change the amplitude, frequency and duration of the pulses emitted from the transducer 12, and a display 22. The coaxial cable 16 is located at the proximal end 23 of the transducer 12, and connects to a connector 17 that is normal to the proximal end 23 and extends outward such that the cable 16 is coaxial to the user's arm.

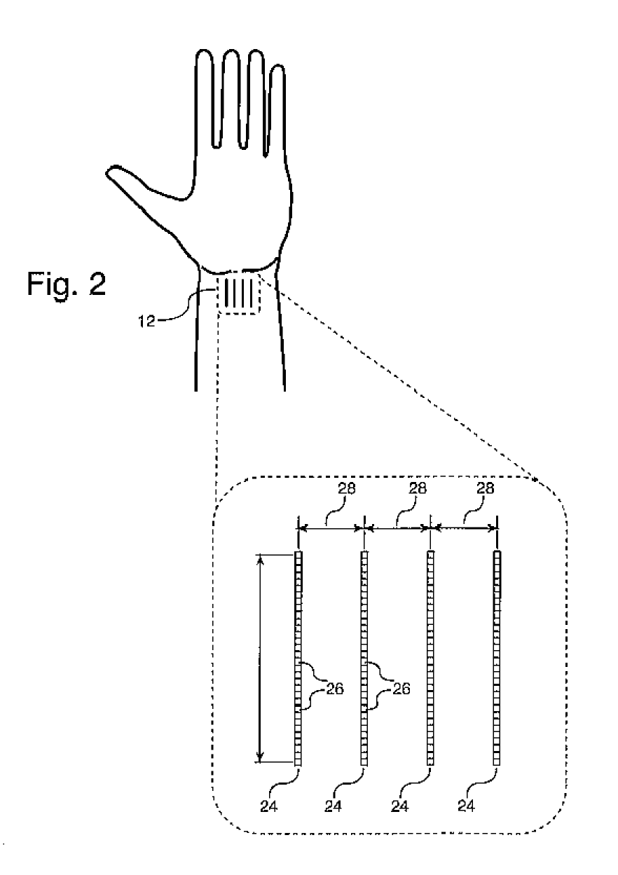

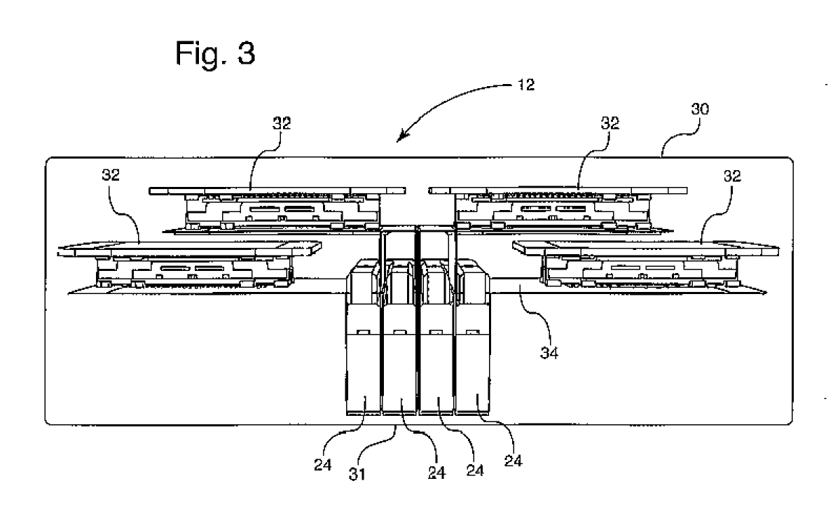

The details of the transducer 12 are shown in FIG. 2. For use with the tendons of the fingers that pass within the wrist, the transducer has four sub-arrays 24, each with 32 elements 26 or preferably eight sub-arrays 24, each with 16 elements. The elements preferably have a cross-sectional size of 100 by 100 microns or 200 by 200 microns. The sub-arrays 24 are parallel to one another to define a gap 28. As shown in FIG. 3, the transducer has a housing 30, with the sub-arrays 24 located at a proximal side 31. Circuit boards 32 are offset in the housing 30 to conserve space and allow the transducer to be small enough for a user to wear. There may be two, three or four or more circuit boards. A flexible ribbon 34 is for electrical communication between the circuit boards 32 and the elements 26. Again, these allow for a compact design.

As would be known to one skilled in the art, if other than a 128 element linear array was used as the starting array, the number of elements 26 in each sub-array 24 would not necessarily be 16 or 32. For example, the number of elements 26 can also vary between about 12 elements 26 to about 32 elements 26 or 16 elements 26 and up to as many as 52 elements 26, and all ranges therebetween. Note that a 128 element array, however arranged into sub-arrays allows for the use of conventional ultrasound technology. The sub-arrays 24 are approximately 3 mm apart (centre to centre) in a four sub-array design and are approximately 1 mm, 1.5 mm, or 2 mm apart in an eight sub-array design. The spacing is selected to allow each sub-array 24 to sit on the skin of the user above a group of tendons, while allowing for some redundancy in imaging the same tendon with multiple sub-arrays 24. As would be known to one skilled in the art, the gap 28 could be larger, for larger users, for example, but not limited to about 3.5 mm to about 5 mm and all ranges therebetween. Similarly, the gap 28 could be smaller for a smaller user, for example, but not limited to about 2.0 mm to about 2.9 mm and all ranges therebetween. The pitch of the elements 26 is preferably no more than about 0.300 mm.

For use with the tendons of the hand, including the tendons within the carpel tunnel and the thumb, a five sub-array transducer with 25 elements per sub-array, or an eight sub-array transducer, with 16 elements per sub-array, or any combination therebetween would be employed. The sub-arrays for the tendons in the carpel tunnel would again be spaced about 3 mm apart, and the sub-arrays for the thumb would be spaced a suitable distance from the other sub-arrays to allow for focusing on the thumb tendons.

For use with prosthetic devices, the transducer has at least two sub-arrays. The tendons will be monitored by the sub-arrays and the computational hardware system. Monitoring the tendons in this way will allow for multi-degree-of-freedom function for advanced prosthetic devices.

For use with exoskeletons, the transducer has at least two sub-arrays. The tendons will be monitored by the sub-arrays and the computational hardware system. Monitoring the tendons in this way will allow for advanced functional control of mechanical exoskeletal devices.

The number of total elements in a transducer, being the sum of all elements of all sub-arrays, controls the speed at which the moving tissue can be scanned. The lower the number of elements, given the same computational hardware, the faster the scanning. As would be known to one skilled in the art, commercial transducers ordinarily have 128 elements, or in some cases 256 elements, in total. The transducer 12 is designed with 128 elements in total, to acquire tissue image frames from all sub-arrays at least 30 frames per second, preferably 40 frames per second or 50 frames per second, more preferable 60 frames per second and up to 200 frames per second if using single-line density, and half that if using double-line density. It is anticipated that one would not want to have less than 12 elements per sub-array as the resolution would decrease below an acceptable level.

The centre frequency is at least about 8.5 MHz, preferably 12 MHz and most preferably 14 MHz, with a bandwidth of about 85-95%. The images from the sub-arrays 24 are sent to the computational hardware and software 18. The parameters allow for images to preferably be collected from a depth of about 2 mm to about 25 mm into the tissue, and all ranges therebetween. The design of the sub-arrays and the software design allows for optimized imaging at or close to the surface of the body. Without being bound to theory, the numerical aperture, F-number, and the focal depth control this. The F-number is the focal depth (mm) divided by the aperture size (mm). The numerical aperture is created by firing a burst of elements in sequential groups of at most twelve, for example, 6 to 18, then 7 to 19, then 8 to 20. After each burst, those elements listen. Each move is no more than 300 microns, preferably 200 microns and most preferably 100 microns. Hence, the elements are at a pitch of about 300 microns, preferably 200 microns and most preferably 100 microns. Both the pitch and the size of the elements control lateral resolution. In addition, the synthetic aperture technique can be used to double the lateral resolution, known as double-line density, where each move is 150 microns, preferably 100 microns, and most preferably 50 microns. Depth resolution is a function of time, and is controlled by the beamformer and the computational hardware.

The computational hardware and software 18 is configured to determine displacement, incremental displacement and velocity. The computational processing hardware 18 is based on a Parallella platform (http://www.parallella.org/introduction/). Parallella is a high performance computing platform the size of a credit card. The software for processing the ultrasound data and obtaining the tendon displacement information is implemented on the Parallella.

The displacement, incremental displacement and velocity is described in U.S. Provisional Patent Application Ser. No. 61/841,156, entitled TISSUE DISPLACEMENT ESTIMATION BY ULTRASOUND SPECKLE TRACKING and filed 28 Jun. 2013, incorporated herein in its entirety by reference. The computational hardware and software may also be configured to provide instructions to permit measuring a stationary region of interest using alternative technology. In addition to gated tracking, the software provides instructions for transforming data from two or more sub-arrays simultaneously, and providing data showing movement of two or more moving parts simultaneously.

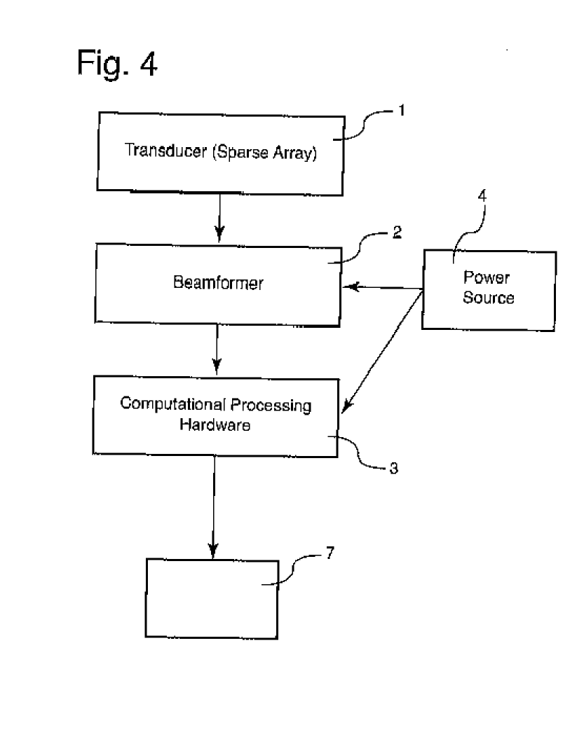

Implementation of the method of the present technology is as follows. With reference to FIG. 4, at steps 1-2, for a given sub-array of 32 elements, up to 12 elements are fired to produce a variable aperture. It is important to note that typical element firing sequence for a 128 element linear array uses a single sized moving aperture. Since the present technology essentially splits a 128 element array into four sub-array sections of 32 elements, the aperture cannot be split across two different sub-arrays. Therefore, a subroutine is implemented that has a variable-sized moving aperture that begins small, then grows to 12 elements, and then tapers in size towards the end of the 32nd element in a sub-array. This reduces the potential for signal cross-talk and other errors which can occur between sub-arrays.

By using these aperture settings, tissue can be focused at 2mm-25mm deep (focal depth or distance). ‘Double line density’ is utilized, which can increase the lateral resolution to 150 microns by having 64 scan lines per sub-array or to about 90 microns using a higher line density of 106 lines.

Using the created sub-routines, the received and processed signals in the beamformer are further processed by filtering and demodulation by the signal processor. Tuned amplifiers are used as filters in order to remove noise. The signal is then demodulated by converting the echo voltages from the beamformer, into a beamformed radio-frequency (rf) signal. The beamformed rf signal is an amplitude-time plot, representing the reflected soundwave along a scan line. This process is repeated, until beamformed rf files from all scan lines for a given sub-array are collected. This process is repeated on all sub-arrays.

For a given sub-array, the beamformed rf signal from Step 2 is further processed, by performing an envelope detection algorithm. For a single scan-line, the envelope of the beamformed rf signal is calculated using a mathematical transform. This way, the negative amplitude peaks are inverted, providing an amplitude value for a window of time (corresponding to the depth). Thus, a series of amplitudes corresponding to the different depths (or windows of time) in the tissue can be calculated. Hence, the data stream now represents the amplitude of the reflected soundwave as a function of depth (or time) along a scan line. This is repeated on all scan lines in a given sub-array, giving a single data-set. Steps 1 through 3 are then repeated to form another data-set from the sub-arrays. The data-sets previously described contain independently collected and processed echoes from the moving tendon, which are separated in time by what is referred to as the sample rate or frame rate. The frame rate should be high enough so that the moving tendon does not displace too much between data-sets. The present technology uses 100 frames-per-second, but could also use as low as 30 frames per second.

The envelop detected rf data of Step 2 is processed in Step 3 to provide tendon tracking at Step 7. Step 3 involves writing low-level algorithms (sub-routines) in order to implement a proper firing sequence of the array elements, collect the reflected soundwave signals (echoes), filter out noise, and storing data in memory. Once the signal is processed at Step 3, an image is provided through image processing at Step 4.

Speckle tracking is used to estimate interframe (one frame to the next frame) musculoskeletal (MSK) displacement in a sequence of consecutive ultrasound images. The present technology uses a method that estimates MSK displacement on a sequence of collected images using a block matching technique. The block matching technique defines a template sub-section in a reference ultrasound image frame. This template sub-section encompasses the desired section of speckle that is to be tracked, and the block matching method searches for a matching block in the subsequent frame. The criteria for determining a suitable match to the template in the subsequent frame utilizes a similarity measure as a comparison metric. Once the match is found, the interframe displacement is calculated.

The following sections describe the auto-location algorithm to locate the placement of the template and region of interest. The speckle tracking algorithm is used on all sub-arrays.

### a) Locating the Ideal Tracking Location

The ideal tracking location is found by repeating steps b) through d) at many locations along the tendon. This creates a displacement field. The ideal template location is the area with the highest displacement in the field. After finding the ideal template location, further analysis such as incremental displacement and velocity is more effectively calculated.

Locating Template and Region of Interest, Interframe Displacement Estimation and Total Displacement Estimation are as described in U.S. Provisional Patent Application Ser. No. 61/841,156, entitled TISSUE DISPLACEMENT ESTIMATION BY ULTRASOUND SPECKLE TRACKING and filed 28 Jun. 2013, incorporated herein in its entirety by reference.

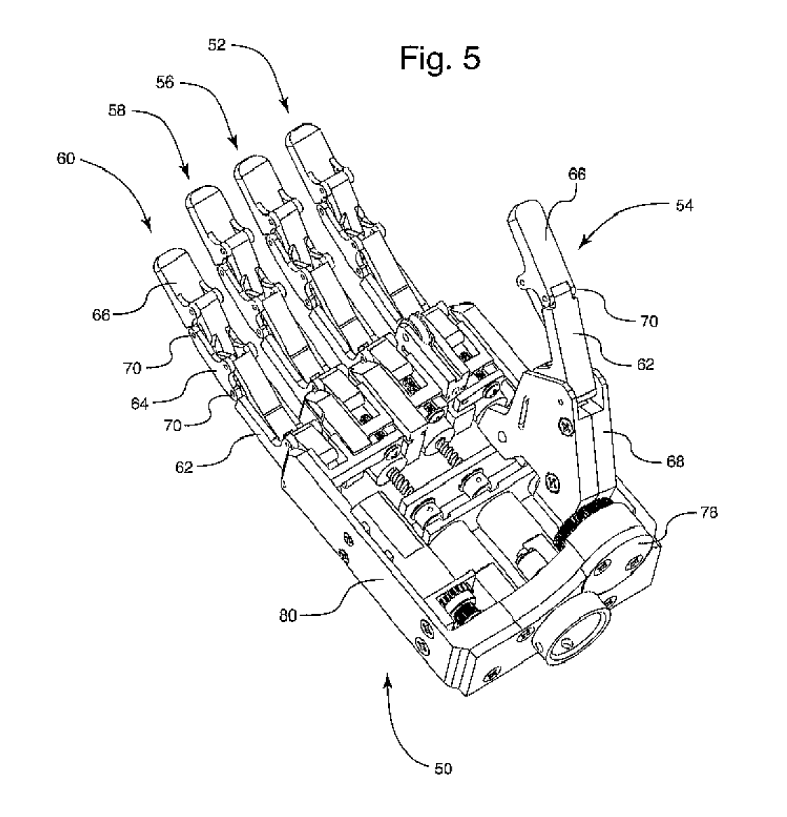

A prosthetic hand, generally referred to as 50, is shown in FIG. 5. The prosthetic hand has 4 degrees of freedom, as follows:

1 motor (i.e. 1 DOF) for index finger and thumb to move together, generally referred to as 52, and thumb, generally referred to as 54 “flex/extend”;

1 motor (i.e. 1 DOF) for middle finger, generally referred to as 56 “flex/extend”;

1 motor (i.e. 1 DOF) for the ring finger, generally referred to as 58 and pinky finger, generally referred to as 60 to move together to “flex/extend”; and

1 motor (i.e. 1 DOF) for the thumb 54 to “adduct/abduct”, for a total of four degrees of freedom.

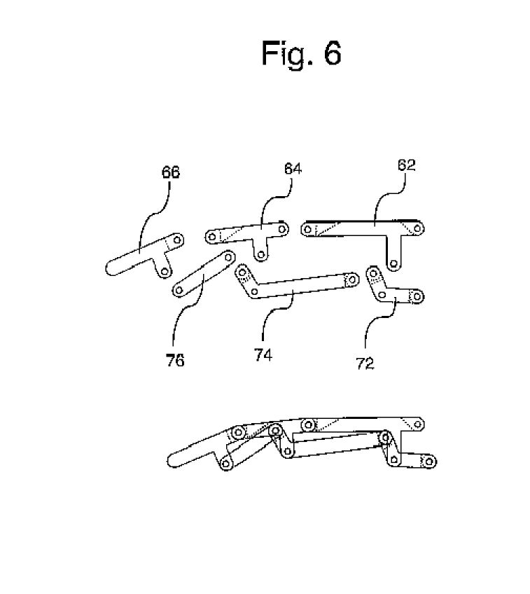

Each finger has a proximal phalanx 62, an intermediate phalanx 64 and a distal phalanx 66. The thumb 54 has a metacarpal 68, a proximal phalanx 62 and a distal phalanx 66. The phalanges, 62, 64 and 66 are hingedly connected by interphalangeal joints 70. As shown in FIG. 6, a proximal link 72, an intermediate link 74 and a distal link 76 are connecting links that help to define the motion of the finger 52, 56, 58, 60. The links 72, 74, 76 are pivotally connected to both the interphalangeal joints 70 and the phalanges 62, 64, 66. Similarly, the thumb 54 has a proximal link 72 and a distal link 76, with the distal link 76 for pivotal connection to the interphalangeal joint 70 and the distal phalanx 66. These joints allow for flexion and extension. Returning to FIG. 5, a pivot assembly 78 connects to a palm plate 80 and the metacarpal 68. The pivot assembly 78 allows for the metacarpal 68, the proximal phalanx 62 and the distal phalanx 66 to flex, extend, abduct and adduct.

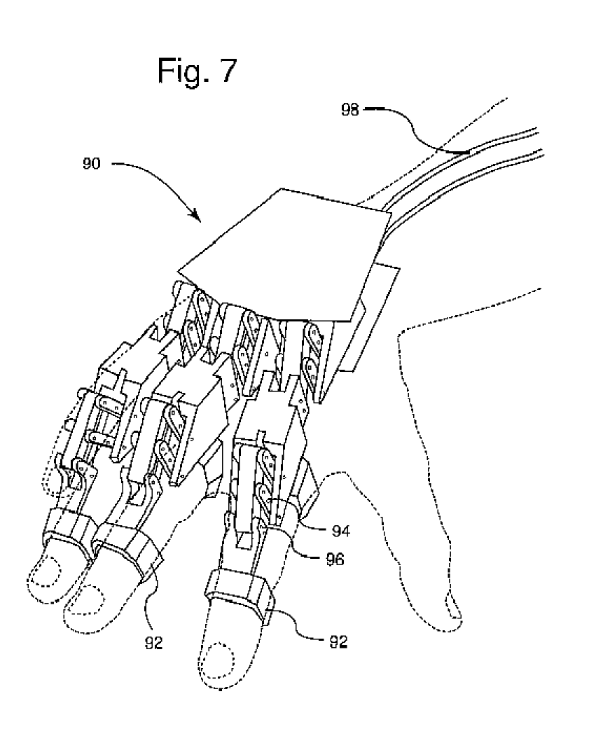

An exoskeleton, generally referred to as 90 is shown in FIG. 7. Cuffs 92 are used to affix linkages 94 to the user's digits. The linkages 94 have pivot joints 96 between them and between the cuffs 92. Control lines 98 extend to the linkages.

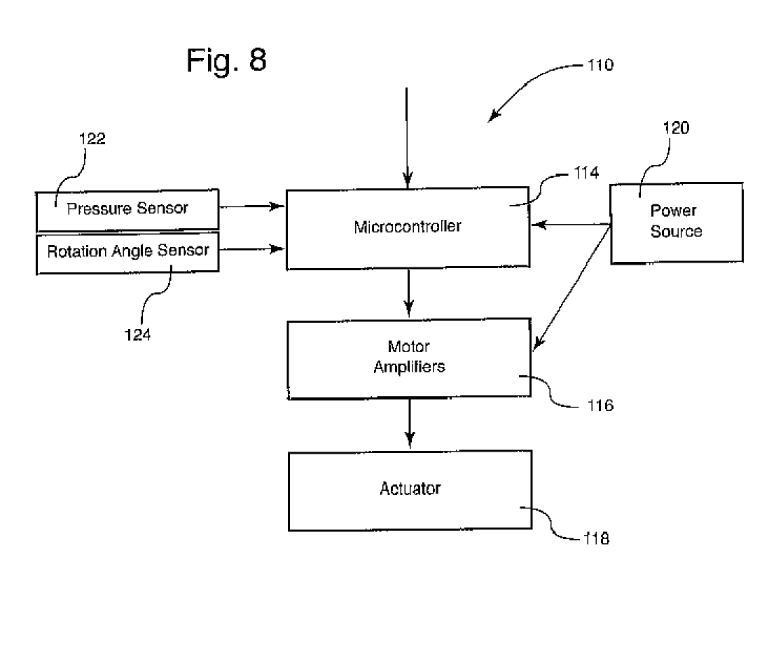

As shown in FIG. 8, the prosthetic 50 or exoskeleton 90 controller 110 is comprised of a compact microcontroller 114, motor amplifiers 116, actuators 118, power source 120, pressure sensors 122 and a rotational angle sensor 124.

The tendon displacement data is then sent to the microcontroller 114. The microcontroller 114 interprets the displacement signal, and converts it into electric signals sent to the motor amplifiers, which drive the actuators 118. Examples of microcontrollers include the Raspberry Pi (http://www.raspberrypi.org/), and Arduino (http://www.arduino.cc/)

The motor amplifiers 116 power the actuators 118.

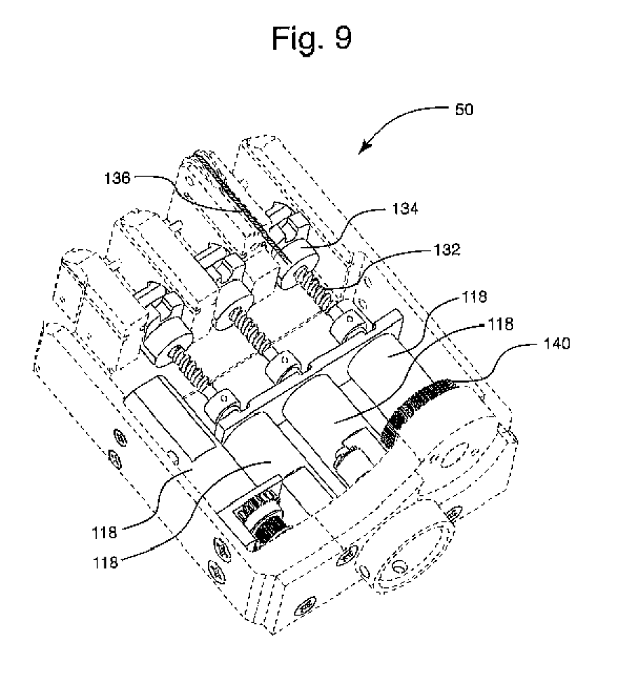

For the prosthetic hand 50, as shown in FIG. 9, there are four actuators 118, each consisting of an electric DC motor, encoder and gearbox, which provides rotational output at an appropriate speed. Three of these actuators 118 are used for flexion and extension of the four fingers and thumb, where these three actuators 118 are connected to lead-screws 132. The lead-screws 132 are connected to sliders 134 which provide linear displacement, that causes the four fingers or thumb to flex or extend. In particular, one actuator 118 will actuate the index finger and thumb together, to flex or extend together, where the thumb is connected to the common slider 134 by a cable 136. One actuator 118 will cause the middle finger to flex and extend. One actuator 118 will cause the ring and pinky fingers to flex and extend, together, where both fingers are connected to a common slider 134. The remaining actuator 118 allows the thumb to adduct or abduct and is connected to the thumb using a cogged belt and pulley system 140. This entire configuration of parts and actuators (except fingers, thumb and cable) fits into the palm of the hand 50, where the palm is 80 mm long,×65 mm wide, by 20 mm deep.

An exoskeleton has a similar configuration.

The microcontroller 114 and amplifiers 116 are powered by a lithium ion battery as the power source 120.

The pressure sensors 122 and rotational angle sensors 124 provide feedback to the microcontroller 114. The pressure sensors 122, also known as touch sensors, are small sensors on the fingertips. These sensors ensure grasps are performed with the appropriate force and pressure. The rotational angle sensor 124, also known as an encoder, provides the microcontroller 114 with information on the location of the fingertips and joint rotation configuration.

The technology can be best understood with reference to the following exemplary examples.

### Example 1

A person is experiencing reduced mobility in their fingers and wants a non-invasive clinical diagnosis. A practitioner has the ultrasound system of the present technology. The transducer is strapped on to the person's wrist, such that the transducer is adjacent the user's tendons, on the underside of the wrist. The person is instructed to open and close their fingers, sequentially and in groupings of one or more fingers. The ultrasound system collects, analyses, displays and stores data on the person. A data set and an image are produced for each sub-array. The results show that one tendon moves at a reduced velocity relative to the other tendons.

### Example 2

A person is experiencing reduced mobility in their fingers and wants a clinical diagnosis. A practitioner has the ultrasound system of the present technology. The transducer is strapped on to the person's wrist, such that the transducer is adjacent the user's tendons, on the underside of the wrist. The person is instructed to type on a keyboard, such that their fingers are used individually. The ultrasound system collects, analyses, displays and stores data on the person. A data set and an image are produced for each sub-array. The transducer is then strapped onto the person's other wrist, without having to reconfigure the transducer and strap as it can be directly transferred from one side to the other. The person is again instructed to type on the keyboard, such that their fingers are used individually. Again the ultrasound system collects, analyses, displays and stores data on the person. The results show that at least one tendon on one side displaces less than at least one tendon on the other side.

### Example 3

A user is rehabilitating a tendon. The user has the ultrasound system of the present technology and has been provided a benchmark of “normal” displacement and velocity by a practitioner, who has previously measured these parameters on the matching tendon and its adjacent tendons on the other side. The user straps the transducer on to the wrist associated with the affected tendon. The user opens and closes their fingers sequentially and in groups of two or more, including closing their hand. The ultrasound system collects, analyses, and compares data on the user. A data set and an image are produced for each sub-array. The output shows that the tendon is improving in at least one of velocity and mobility.

### Example 4

A person is experiencing reduced mobility in their hand and wants a non-invasive clinical diagnosis. A practitioner has the ultrasound system of the present technology. The transducer is strapped on to the person's wrist, such that the transducer is adjacent the user's finger tendons, on the underside of the wrist and the thumb tendon. The person is instructed to open and close their fingers and thumb, sequentially and in groupings of two or more fingers. Each sub-array provides a signal. The ultrasound system collects, analyses, displays and stores data on the person. A data set and an image are produced for each sub-array. The ultrasound system collects, analyses, displays and stores data on the person. The results show that one tendon moves at a reduced velocity relative to the other tendons.

### Example 5

A prosthetic device 50 is an electro-mechanical system worn by a disabled user with hand or finger loss, to restore their functionality. Such devices are comprised of the ultrasound system 10, the controller 110, and the prosthetic 50. The transducer 12 of the ultrasound system 10 detects the remnant tendon's motion in the wrist. A data set and an image are produced for each sub-array 24, hence a plurality of data sets are produced. This allows for simultaneous measurement of multiple tendons, or allows for redundant measurement of a single tendon. The ultrasound system 10 collects and analyses the displacement data in real-time, and forwards the data to the microcontroller 114. The microcontroller 114 provides the control signals to amplifiers 114. The amplifiers 116 drive the actuators 118 that move the prosthetic 50 to perform the desired task.

### Example 6

An exoskeleton 90 is an electro-mechanical structure worn by a person to augment their strength in performing various activities. Such structures are usually worn by disabled users, to perform tasks where his/her muscles are insufficiently strong to carry out the activity. Such structures are also proposed for healthy individuals, to carry out tasks that require strength beyond ordinary human strength. The exoskeleton may be worn by the user, or may be part of a robot. The transducer 12 is strapped on, or otherwise affixed to the location to be tracked, for example wrist. The person moves their body, and the tendon or tendons located in the wrist have their displacement measured by the transducer 12. A plurality of sub-arrays 24 are employed in the transducer. A data set and an image are produced for each sub-array, hence a plurality of data sets are produced. This allows for simultaneous measurement of multiple tendons, or allows for redundant measurement of a single tendon. The ultrasound system 10 collects and analyses the displacement data in real-time, and forwards the data to the microcontroller 114. The microcontroller 114 provides the control signals to amplifiers 116. The amplifiers 116 drive the actuators 118 that move the exoskeleton 90 to perform the desired task.

Advantages of the exemplary embodiments described herein may be realized and attained by means of the instrumentalities and combinations particularly pointed out in this written description. It is to be understood that the foregoing general description and the following detailed description are exemplary and explanatory only and are not restrictive of the claims below. While example embodiments have been described in detail, the foregoing description is in all aspects illustrative and not restrictive. It is understood that numerous other modifications and variations can be devised without departing from the scope of the example embodiment.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize, or be able to ascertain using no more than routine experimentation, many equivalents to the specific example embodiments specifically described herein. Such equivalents are intended to be encompassed in the scope of the claims, if appended hereto or subsequently filed.

1. An ultrasound tracking system for tracking internal structures by acquiring and processing two-dimensional images, the system comprising a transducer, a beamformer, and computational processing hardware, wherein the transducer has a plurality of sub-arrays with a gap between adjacent sub-arrays, the sub-arrays in generally parallel relation to one another, the sub-arrays comprising at least 12 elements, the beamformer in electronic communication with the sub-arrays, and the computational processing hardware comprising instructions for transforming signals from the sub-arrays into a plurality of data sets.

2. The ultrasound tracking system of claim 1, wherein there are at least three sub-arrays, each comprising at least about 16 elements.

3. The ultrasound tracking system of claim 2, wherein the elements have a pitch of no more than about 300 microns.

4. The ultrasound tracking system of claim 3, wherein the gap between adjacent sub-arrays is less than about 3 millimeters.

5. The ultrasound tracking system of claim 4, wherein there are four sub-arrays, each comprising 32 elements.

6. The ultrasound tracking system of claim 5, wherein the transducer further comprises at least two circuit boards, the circuit boards being offset to provide a compact transducer.

7. The ultrasound tracking system of claim 6, further comprising a cable for communication between the transducer and the computational processing hardware, the cable extending normal to a proximal end of the transducer.

8. The ultrasound tracking system of claim 7, wherein the beamformer and computational processing hardware further comprises instructions for sequential firing of the elements.

9. The ultrasound tracking system of claim 8, wherein the beamformer and computational processing hardware further comprise instructions for measuring a stationary region of interest.

10. A transducer for use with an ultrasound system, the transducer comprising: at least two sub-arrays in parallel relation to define an at least one gap between the sub-arrays, the sub-arrays comprising an at least 12 elements, the elements having a pitch of at most about 300 microns; an at least two circuit boards; a housing for housing the sub-arrays and the circuit boards; and a connector for connecting a cable, the connector located on a proximal side of the housing and extending normal to the proximal side.

11. The transducer of claim 10, the transducer comprising four sub-arrays in generally parallel relation to define three gaps of at most about 3 mm and four circuit boards, the circuit boards being offset.

12. The transducer of claim 11, wherein the sub-arrays comprise 32 elements.

13. An active hand prosthesis to provide four degrees of freedom, the active hand prosthesis comprising a hand prosthesis and a controller, the hand prosthesis comprising: at least an index finger, a middle finger, and a ring finger, each finger comprising: a proximal phalanx, an intermediate phalanx and a distal phalanx, each hinged at an interphalangeal joint; and linkages to an actuator; a thumb comprising: a metacarpal; a proximal phalanx; and a distal phalanx; the proximal phalanx and the distal phalanx hinged at an interphalangeal joint, the metacarpal pivotally attached to a palm plate by a pivot assembly, the pivot assembly in communication with a pivot assembly actuator, to provide adduction and abduction; and linkages to the index finger actuator; a controller, the controller comprising: a microcontroller, motor amplifiers, the actuators, and a power source; and a tendon tracking system in communication with the microcontroller.

14. The active hand prosthesis of claim 13, comprising four actuators, each comprising an electric direct current (DC) motor, an encoder and a gearbox, wherein three actuators: are configured to effect flexion and extension of the index finger, the middle finger, the ring finger, a pinky finger and a thumb; and are each connected to a lead-screw, each lead-screw connected to a slider, and a fourth actuator is connected to the thumb with a cogged belt and pulley system.

15. The active hand prosthesis of claim 14, wherein the index finger is connected to a first common slider that is common with the thumb by a cable to actuate the index finger and thumb together, and the ring finger is connected to a second common slider that is common with the pinky finger, to actuate the ring finger and pinky finger together.

16. The active hand prosthesis of claim 15, further comprising pressure sensors and a rotational angle sensor.

17. The active hand prosthesis of claim 16, wherein the tracking system is an ultrasound tracking system.

18. The active hand prosthesis of claim 17, wherein the ultrasound tracking system is a sparse array ultrasound tracking system.

19. The active hand prosthesis of claim 18, wherein the sparse array ultrasound tracking system comprises a transducer, the transducer comprising a plurality of sub-arrays with a gap between adjacent sub-arrays, the sub-arrays in generally parallel relation to one another, the sub-arrays comprising an at least 12 elements.

20. The active hand prosthesis of claim 19, wherein there are at least three sub-arrays, each comprising at least about 12 elements.
